(19) United States
(12) Patent Application Publication
(10) Pub. No.: US 2024/0197743 A1
(43) Pub. Date: Jun. 20, 2024

(54) WEE1 COMPOUND FOR TREATING UTERINE SEROUS CARCINOMA

(71) Applicant: Recurium IP Holdings, LLC, San Diego, CA (US)

(72) Inventors: Philippe Pultar, Windham, NY (US); Dimitrios Voliotis, Madison, NJ (US); Marcus Rodrigues Viana, Seekonk, MA (US); Fernando Donate, Santa Monica, CA (US); Petrus Rudolf de Jong, San Diego, CA (US); Ahmed Abdi Samatar, West Windsor, NJ (US); Kevin Duane Bunker, Escondio, CA (US); Peter Qinhua Huang, San Diego, CA (US)

(21) Appl. No.: 18/554,951

(22) PCT Filed: Apr. 8, 2022
(86) PCT No.: PCT/US2022/024079

§ 371 Date: Oct. 11, 2023

Related U.S. Application Data
(60) Provisional application No. 63/174,004, filed on Apr. 12, 2021.
(60) Provisional application No. 63/174,005, filed on Apr. 12, 2021.

(51) Int. Cl. A61K 31/519; A61K 9/00; A61P 35/00

(52) U.S. Cl. CPC A61K 31/519; A61K 9/0053; A61P 35/00

(57) ABSTRACT

Disclosed herein is a method for treating a subject having uterine serous carcinoma with a WEE1 inhibitor, or a pharmaceutically acceptable salt thereof.

### INCORPORATION BY REFERENCE TO PRIORITY APPLICATIONS

This application claims priority to U.S. Provisional Application Ser. No. 63/174,004, filed Apr. 12, 2021, and U.S. Provisional Application Ser. No. 63/174,005, filed Apr. 12, 2021, both of which are incorporated herein by reference in their entireties.

### BACKGROUND

### Field

The present application relates generally to methods of using a WEE1 inhibitor to treat uterine serous carcinoma (USC).

### Description

DNA is constantly damaged from the environment. Light, chemicals, stress, and cellular replication lead to single- or double-stranded breakage along DNA's backbone. Typically, organisms defend against DNA damage by repair proteins that either re-connect or re-synthesize damaged DNA. The correct functioning of these proteins is essential for life. The incorrect replacement of nucleotides into DNA can cause mutations (and other genetic alterations including but not limited to insertions, deletions, and frameshifts), genetic disease, and loss of protein function. The altogether loss of DNA repair can cause cell death, tumor progression, and cancer.

Cell cycle checkpoints are important for proper DNA repair, ensuring that cells do not progress with cellular replication until their genomic integrity is restored. WEE1 is a nuclear kinase involved in the G2-M cell-cycle checkpoint arrest for DNA repair before mitotic entry. Normal cells repair damaged DNA during G1 arrest. Cancer cells often have a deficient G1-S checkpoint and depend on a functional G2-M checkpoint for DNA repair. WEE1 is overexpressed in various cancer types, and a number of inhibitors and/or degraders of WEE1 are known to those skilled in the art. See, e.g., WO 2019/173082 and WO 2020/069105.

Uterine serous carcinoma (USC) is a highly aggressive Type II endometrial cancer. See, e.g., Ferriss J S et al, Uterine serous carcinoma: key advances and novel treatment approaches. International Journal of Gynecologic Cancer 2021; 31:1165-1174. A phase II study of adavosertib in recurrent uterine serous carcinoma has been reported. See Liu J F et al. Phase II study of the WEE1 inhibitor adavosertib in recurrent uterine serous carcinoma. J Clin Oncol 2021; 39. However, existing therapeutic approaches remain limited, and thus there is an urgent need for additional therapies.

### SUMMARY

Various embodiments provide a method of treating a subject having uterine serous carcinoma (USC), the method comprising administering a therapeutically effective amount of ZN-c3, or a pharmaceutically acceptable salt thereof, to the subject in accordance with a dosing regimen.

These and other embodiments are described in greater detail below.

### BRIEF DESCRIPTION OF THE DRAWINGS

FIG. 1 illustrates certain baseline (November 2020) and 2nd restaging (April 2021) peritoneum imaging results for subject #1, demonstrating confirmed partial response at that follow-up timepoint.

FIG. 2 illustrates certain baseline (January 2021) and 1st restaging (March 2021) peritoneum imaging results for subject #2, demonstrating partial response at that follow-up timepoint.

FIG. 3 illustrates certain baseline (April 2021) and 5th restaging (November 2021) peritoneum imaging results for subject #3, demonstrating complete response at that follow-up timepoint.

FIG. 4 illustrates certain baseline (April 2021) and 5th restaging (November 2021) peritoneum imaging results for subject #3, demonstrating complete response at that follow-up timepoint.

### DETAILED DESCRIPTION

WEE1 is a tyrosine kinase that is a critical component of the ATR-mediated G2 cell cycle checkpoint control that prevents entry into mitosis in response to cellular DNA damage. ATR phosphorylates and activates CHK1, which in turn activates WEE1, leading to the selective phosphorylation of cyclin-dependent kinase 1 (CDK1) at Tyr15, thereby stabilizing the CDK1-cyclin B complex and halting cell-cycle progression. This process confers a survival advantage by allowing tumor cells time to repair damaged DNA prior to entering mitosis. Inhibition of WEE1 abrogates the G2 checkpoint, promoting cancer cells with DNA damage to enter into unscheduled mitosis and undergo cell death via mitotic catastrophe. WEE1 inhibition and/or degradation has the potential to sensitize tumors to DNA-damaging agents, such as cisplatin, and to induce tumor cell death.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The terms “WEE1 inhibition”, “WEE1 inhibitor” and similar terms as used herein refer to inhibiting the activity or function of a WEE1 tyrosine kinase, e.g., by degrading WEE1 tyrosine kinase and/or by reducing the activity of WEE1 tyrosine kinase with regard to mediating phosphorylation of CDK1. A WEE1 inhibitor that functions by degrading WEE1 tyrosine kinase may be referred to herein as a WEE1 degrader.

As used herein, a “subject” refers to an animal that is the object of treatment, observation or experiment. The subject animal may be a mammal such as, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult.

As used herein, the terms “treat,” “treating.” “treatment.” “therapeutic.” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound (e.g. ZN-c3 or pharmaceutically acceptable salt thereof), that elicits the biological or medicinal response indicated. For example, a therapeutically effective amount of such a ZN-c3 compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the ZN-c3 compound, salt or composition required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a ZN-c3 compound, salt or composition, may be the amount that results in: (a) the reduction, alleviation or disappearance of one or more symptoms caused by USC, (b) the reduction of tumor size, (c) the elimination of the tumor, and/or (d) long-term disease stabilization (growth arrest) of the tumor. As an example, an effective amount of a ZN-c3 compound, salt or composition may be the amount which results in the reduction in WEE1 activity and/or phosphorylation (such as phosphorylation of CDC2). The reduction in WEE1 activity is known to those skilled in the art and can be determined by the analysis of WEE1 intrinsic kinase activity and downstream substrate phosphorylation.

As used herein, the term “dosing regimen” refers to the manner in which the ZN-c3 compound is administered to the subject, including route of administration, amount of dose and dosing interval. A dosing regimen may comprise a “periodic” dosing phase, during which a particular dosage amount (e.g., 300 mg) is administered at regular intervals (e.g., once per day) for a particular period of time (e.g., three days). A dosing regimen may further comprise an “intermittent” dosing phase, during which one or more dosing parameters such as dosage amount and/or dosage interval are varied or changed. For example, an intermittent dosing phase may comprise a “rest” phase during which the ZN-c3 compound is not administered or is administered at a reduced dosage amount and/or less frequently.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### WEE1 Compound

ZN-c3 is a selective, orally bioavailable small molecule inhibitor of WEE1, a crucial component of the G2/M cell cycle checkpoint, which prevents cells from entering mitosis to allow repair of DNA damage. ZN-c3 has demonstrated significant in vitro antitumor activity in multiple cell lines and xenograft models.

| ZN-c3 Chemical Structure | ZN-c3 Chemical Name |
|---|---|
| 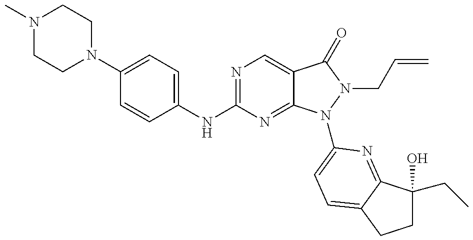 [SMILES: C=CCn1c(=O)c2cnc(Nc3ccc(N4CCN(C)CC4)cc3)nc2n1-c1ccc2c(n1)[C@@](O)(CC)CC2] | (R)-2-allyl-1-(7-ethyl-7-hydroxy-6,7- dihydro-5H-cyclopenta[b]pyridin-2-yl)-6- ((4-(4-methylpiperazin-1-yl)phenyl)amino)- 1,2-dihydro-3H-pyrazolo[3,4-d]pyrimidin- 3-one |

ZN-c3 can be prepared as described in WO 2019/173082 (see, e.g., Example 9B therein), which is hereby incorporated herein by reference and particularly for the purposes of describing methods for making ZN-c3, as well as for making salts and pharmaceutical compositions thereof.

### Pharmaceutical Compositions

Some embodiments described herein relate to a pharmaceutical composition, that can include an effective amount of the ZN-c3 compound, or a pharmaceutically acceptable salt thereof, and a pharmaceutically acceptable carrier, diluent, excipient or combination thereof.

The term “pharmaceutical composition” refers to a mixture of ZN-c3 and/or pharmaceutically acceptable salts thereof, with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the ZN-c3 compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the ZN-c3 active ingredients is typically contained in an amount effective to achieve its intended purpose, and may be provided as a salt with pharmaceutically compatible counterions.

Multiple art-recognized techniques of administering a ZN-c3 compound, salt and/or composition may be used, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, ZN-c3, or a pharmaceutically acceptable salt thereof, can be administered orally.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that include ZN-c3 and/or salt, formulated in a compatible pharmaceutical carrier as described herein, may also be prepared, placed in an appropriate container, and labeled for treatment of an indicated condition.

### Uses and Methods of Treatment

Some embodiments described herein relate to a method of treating a subject having USC, the method comprising administering a therapeutically effective amount of ZN-c3, or a pharmaceutically acceptable salt thereof, to the subject in accordance with a dosing regimen.

Some embodiments described herein relate to the use of a therapeutically effective amount of ZN-c3, or a pharmaceutically acceptable salt thereof, in the manufacture of a medicament for treating a subject having USC in accordance with a dosing regimen. Other embodiments relate to ZN-c3 for use in treating a subject having USC, the treatment comprising administering a therapeutically effective amount of ZN-c3, or a pharmaceutically acceptable salt thereof, to the subject in accordance with a dosing regimen. In various embodiments, the dosing regimen comprises an oral administration of the ZN-c3 to the subject.

In various embodiments, the therapeutically effective amount of the ZN-c3 administered to the subject is ≥300 mg per day. For example, in some embodiments, the therapeutically effective amount of the ZN-c3 administered to the subject is about 300 mg per day. In other embodiments, the therapeutically effective amount of the ZN-c3 administered to the subject is about 350 mg per day. In other embodiments, the therapeutically effective amount of the ZN-c3 administered to the subject is about 200 mg per day. In some embodiments, the therapeutically effective amount of the ZN-c3 administered to the subject is ≥300 mg once daily (QD). In an embodiment, the therapeutically effective amount of the ZN-c3 administered to the subject is about 300 mg once daily (QD). In another embodiment, the therapeutically effective amount of the ZN-c3 administered to the subject is about 350 mg once daily (QD). In still another embodiment, the therapeutically effective amount of the ZN-c3 administered to the subject is about 200 mg once daily (QD). In some embodiments, the therapeutically effective amount of the ZN-c3 administered to the subject is ≥150 mg two times a day (BID). In an embodiment, the therapeutically effective amount of the ZN-c3 administered to the subject is about 150 mg two times a day (BID). In another embodiment, the therapeutically effective amount of the ZN-c3 administered to the subject is about 175 mg two times a day (BID). In still another embodiment, the therapeutically effective amount of the ZN-c3 administered to the subject is about 100 mg two times a day (BID).

In various embodiments, the dosing regimen comprises a periodic dosing phase during which the amount of the dose and the frequency of ZN-c3 administration to the subject is fixed for a period of time, e.g., 3 or more consecutive days. For example, in an embodiment, the periodic dosing phase comprises a daily dose in the range of about 200 mg per day to about 350 mg per day that is fixed for a period of at least three consecutive days. In an embodiment, the daily dose is administered once daily (QD). In another embodiment, the daily dose is administered in divided doses provided two, three or four times daily. In an embodiment, the periodic dosing phase comprises once daily dosing for a period of at least three consecutive days.

In various embodiments, the dosing regimen comprises an intermittent dosing phase during which the amount of the dose and the frequency of ZN-c3 administration to the subject is changed. In an embodiment, the intermittent dosing phase comprises at least one change in amount of the ZN-c3 administered to the subject on a daily basis. In an embodiment, the intermittent dosing phase comprises at least one rest day, e.g., 1, 2, 3, 4, 5, 6 or 7 rest days. For example, in an embodiment, the dosing regimen comprises a periodic phase during which the ZN-c3 is administered to the subject orally on a once daily basis for five days, following by an intermittent or rest phase during which the ZN-c3 is not administered for two days. Such a dosing regimen, which may be referred to as 5 days on/2 days off, may be repeated or cycled as often as needed, depending on the particular case.

In various embodiments, the subject is a human. In some embodiments, the subject has USC that is advanced USC. In some embodiments, the subject has USC that is recurrent USC.

In various embodiments, the subject has been previously treated for the USC by a prior therapeutic regimen. Various therapeutic regimens are known to those skilled in the art or under active development. In an embodiment, the prior therapeutic regimen can include administering a prior USC therapy to the subject. For example, in various embodiments, the prior USC therapy can include at least one selected from Carboplatin, Paclitaxel. Bevacizumab, Trastuzumab, Pembrolizumab, Lenvatinib and Doxorubicin.

The amount of the compound of ZN-c3, or pharmaceutically acceptable salt thereof, that is effective in treating a particular case of USC may vary not only with the particulars of the compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the ZN-c3 compound, salt or composition in amounts that exceed, or even far exceed, the dosage ranges described herein in order to effectively and aggressively treat particularly aggressive cases of USC.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular ZN-c3 compound, salt or composition employed and the specific use for which these therapies are employed. The determination of effective dosage levels, that is the dosage levels necessary to achieve the desired result, can be accomplished by one skilled in the art using routine methods, for example, human clinical trials, in vivo studies and in vitro studies. For example, useful dosages of ZN-c3, or pharmaceutically acceptable salts thereof, can be determined by comparing their in vitro activity, and in vivo activity in animal models. Such comparison can be done by comparison against an established drug, such as carboplatin and/or paclitaxel.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

ZN-c3 compounds, salts and compositions disclosed herein can be evaluated for efficacy and toxicity using known methods. For example, the toxicology of ZN-c3 may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Examples 1-3

### Methods

ZN-c3-001 (NCT04158336) is an open label, multicenter, Phase I dose escalation and expansion study evaluating the safety and clinical activity of ZN-c3 in subjects with advanced or metastatic solid tumors, refractory to standard therapy or for whom no standard therapy is available. Oral, daily ZN-c3 dosing was escalated from 25 mg to 450 mg once daily (QD), with a recommended phase 2 dose of 300 mg QD. Here we report preliminary safety and efficacy results for subjects with USC who received ZN-c3 at doses ≥300 mg QD.

### Results

As of Jan. 21, 2022, a total of 80 subjects were enrolled across all tumor types. For uterine serous carcinoma (USC), there were 14 subjects who received ZN-c3 at doses ≥300 mg QD and were evaluated for safety. Median duration of treatment was 12.4 weeks (range, 2-36 weeks). The safety profile for this subgroup is comparable to the overall patient population with 12 of 14 subjects reporting at least one treatment-related adverse event (TRAE). TRAEs grades 1-4 occurring in ≥3 subjects, by decreasing frequency, included nausea, vomiting, diarrhea, fatigue, decreased appetite, dehydration, and anemia. Grade ≥3 TRAEs occurring in ≥2 subjects included anemia and thrombocytopenia. These hematologic events were limited to 2 subjects each. Eleven of the 14 subjects had measurable disease and at least 1 post-baseline tumor assessment; 3 subjects (27.3%) had confirmed partial response (one of these subjects achieved complete response at the last tumor assessment), 7 (63.6%) had stable disease and 1 (9.1%) had progressive disease as their best overall response. Disease control rate, defined as those with complete response, partial response, and stable disease, was 90.9%.

### USC Confirmed Partial Response #1—Summary

72-year-old, White female, Stage IV USC; metastases to peritoneum and lymph node, ECOG PS 1
2 prior lines of therapy in the advanced/metastatic setting
ZN-c3 starting dose: 350 mg QD in December 2020
Tumor marker CA-125 decreased from 36 U/mL at baseline to a minimum of 12 U/mL after ˜3 months of treatment
Confirmed PR with 49% reduction overall
The subject remained on the study drug for 199 days until radiologic disease progression

| Intent of Prior Treatment | Regimen | Start / Stop |
|---|---|---|
| Advanced/Metastatic | Carboplatin/ | December 2018/ |
| | Paclitaxel | March 2019 |
| Advanced/Metastatic | Paclitaxel/ | December 2019/ |
| | Bevacizumab | March 2020 |
| Maintenance | Bevacizumab | April 2020/ |
| | | October 2020 |

### Tumor Assessment for #1

| | Baseline | Follow-up 1 | Follow-up 2 | Follow-up 3 |
|---|---|---|---|---|
| Tumor Assessment | (November 2020) | (February 2021) | (April 2021) | (June 2021) |
| Target Lesions | 24 mm | 15 mm | 16 mm | 16 mm |
| Spleen | 21 mm | 8 mm | 7 mm | 7 mm |
| Peritoneum | Sum: 45 mm | Sum: 23 mm (PR) | Sum: 23 mm (PR) | Sum: 23 mm (PR) |
| New Lesions | Baseline | No | No | No |
| Percentage of | Baseline | −48.9% | −48.9% | −48.9% |
| overall tumor | | | | |
| reduction | | | | |
| Tumor Response by | Baseline | Partial | Partial | Progressive |
| RECIST 1.1 | | Response | Response | Disease |

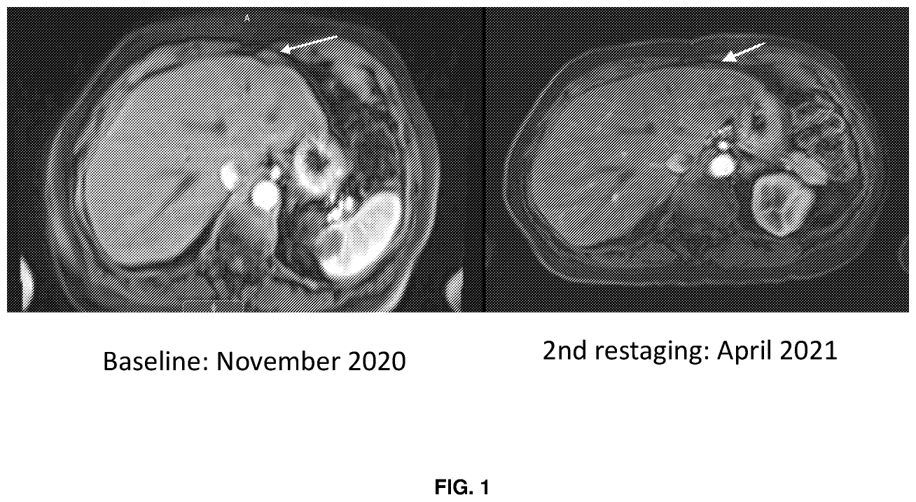

FIG. 1 illustrates certain baseline (November 2020) and 2nd restaging (April 2021) peritoneum imaging results for subject #1, demonstrating confirmed partial response at that follow-up timepoint.

### USC Partial Response #2—Summary

69-year-old, African American female, Stage IV USC; metastases to lymph node and lung, ECOG PS 0
4 prior lines of therapy in the advanced/metastatic setting, including prior use of pembrolizumab plus lenvatinib
ZN-c3 starting dose: 300 mg QD in January 2021
Tumor marker CA-125 decreased from 440 U/mL at baseline to <50 U/mL after 2 months of treatment
Confirmed PR with 43% reduction overall
The subject remained on the study drug for 230 days until radiologic disease progression

### Previous Therapy Experience for #2

| Intent of Prior Treatment | Regimen | Start / Stop |
|---|---|---|
| Advanced/Metastatic | Carboplatin/ | September 2019/ |
| | Paclitaxel/ | November 2019 |
| | Bevacizumab | |
| Advanced/Metastatic | Trastuzumab | February 2020/ |
| | | April 2020 |
| Advanced/Metastatic | Pembrolizumab/ | April 2020/ |
| | | May 2020 |
| | Lenvatinib | |
| Advanced/Metastatic | Doxorubicin | June 2020/ |
| | Liposomal | November 2020 |

### Tumor Assessment for #2

| | Baseline | Follow-up 1 | Follow-up 2 |
|---|---|---|---|
| Tumor Assessment | (January 2021) | (March 2021) | (April 2021) |
| Target Lesions | 15 mm | 06 mm | 07 mm |
| Lymph Node | 15 mm | 11 mm | 10 mm |
| Lymph Node | Sum: 30 mm | Sum: 17 mm (PR) | Sum: 17 mm (PR) |
| New Lesions | Baseline | No | No |
| Percentage of | Baseline | −43% | −43% |
| overall tumor | | | |
| reduction | | | |
| Tumor Response by | Baseline | Partial Response | Partial Response |
| RECIST 1.1 | | | |

| | Follow-up 3 | Follow-up 4 | Follow-up 5 |
|---|---|---|---|
| Tumor Assessment | (June 2021) | (July 2021) | (August 2021) |
| Target Lesions | 8 mm | 11 mm | 11 mm |
| Lymph Node | 10 mm | 10 mm | 10 mm |
| Lymph Node | Sum: 18 mm (PR) | Sum: 21 mm (PR) | Sum: 21 mm (PR) |
| New Lesions | No | No | No |
| Percentage of | −40% from baseline | −30% from baseline | −30% from baseline |
| overall tumor | +6% from nadir | +24% from nadir | +24% from nadir |
| reduction | | | |
| Tumor Response by | Partial Response | Partial Response | Progressive Disease |
| RECIST 1.1 | | | |

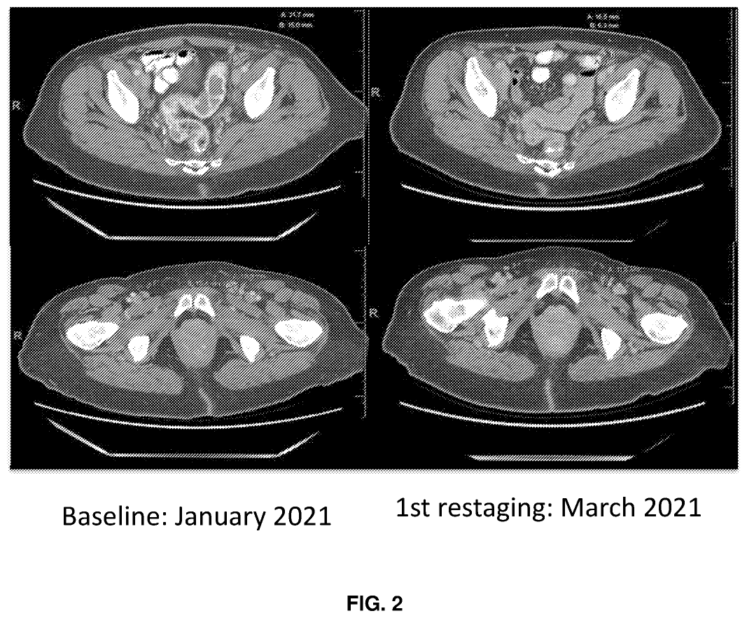

FIG. 2 illustrates certain baseline (January 2021) and 1st restaging (March 2021) peritoneum imaging results for subject #2, demonstrating partial response at that follow-up timepoint. The partial response was confirmed at the subsequent tumor assessment.

### USC Partial Response #3—Summary

60-year-old, White female, Stage IV USC; metastases to peritoneum, ECOG PS 1
2 prior lines of therapy in the advanced/metastatic setting, including prior use of pembrolizumab plus lenvatinib
ZN-c3 starting dose: 300 mg QD in April 2021
Tumor marker CA-125 was not collected at baseline
Unconfirmed Complete Response with 100% reduction overall
The subject remained on the study drug for 252 days until clinical disease progression

### Previous Therapy Experience for #3

| Intent of Prior Treatment | Regimen | Start / Stop |
|---|---|---|
| Advanced/Metastatic | Carboplatin/ | August 2019/ |
| | Paclitaxel | January 2020 |
| Advanced/Metastatic | Pembrolizumab/ | September 2020/ |
| | Lenvatinib | February 2021 |

### Tumor Assessment for #3

| | Baseline | Follow-up 1 | Follow-up 2 |
|---|---|---|---|
| Tumor Assessment | (April 2021) | (May 2021) | (July 2021) |
| Target Lesions | 36.6 mm | 21.7 mm | 12.5 mm |
| Peritoneum | 17.8 mm | 15.3 mm | 5 mm (too small) |
| Peritoneum | Sum: 54.4 mm | Sum: 37 mm (PR) | Sum: 17.5 mm (PR) |
| New Lesions | Baseline | No | No |
| Percentage of | Baseline | −32% | −67.8% |
| overall tumor | | | |
| reduction | | | |
| Tumor Response by | Baseline | Partial Response | Partial Response |
| RECIST 1.1 | | | |

| | Follow-up 3 | Follow-up 4 | Follow-up 5 |
|---|---|---|---|
| Tumor Assessment | (August 2021) | (September 2021) | (November 2021) |
| Target Lesions | 7.8 mm | 5 mm (too small) | 0 mm |
| Peritoneum | 5 mm (too small) | 5 mm (too small) | 0 mm |
| Peritoneum | Sum: 12.8 mm (PR) | Sum: 10 mm (PR) | Sum: 0 mm (CR) |
| New Lesions | No | No | No |
| Percentage of | −76.5% | −82% | −100% |
| overall tumor | | | |
| reduction | | | |
| Tumor Response by | Partial Response | Partial Response | Complete Response |
| RECIST 1.1 | | | |

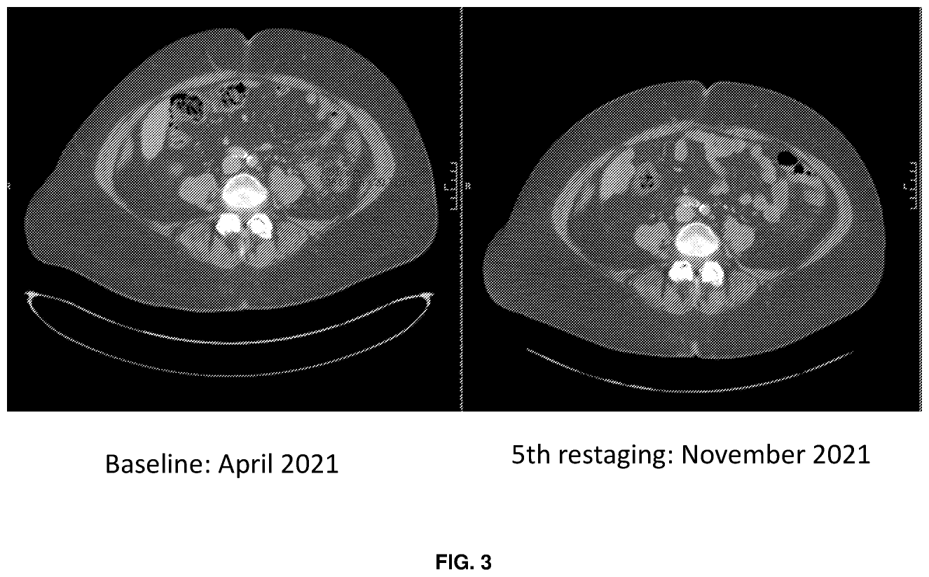

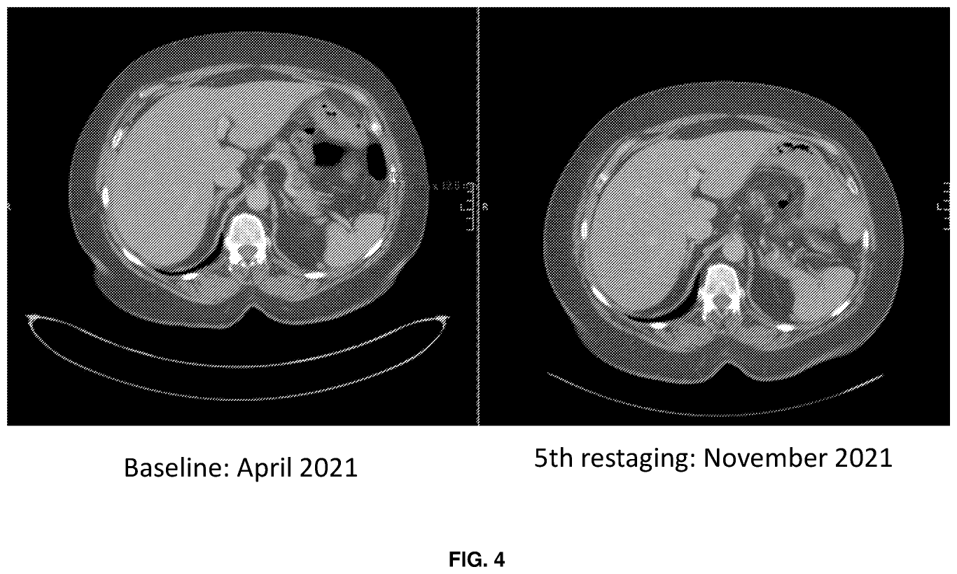

FIGS. 3 and 4 illustrate certain baseline (April 2021) and 5th restaging (November 2021) peritoneum imaging results for subject #3, demonstrating complete response at that follow-up timepoint.

### CONCLUSION

ZN-c3 appears to be safe and well-tolerated as a single agent at oral doses ≥300 mg QD and demonstrated clinical activity in subjects with recurrent or advanced USC as exemplified in Examples 1-3. Safety results in these subjects were consistent with those of the full study population. The ongoing dose expansion study is enrolling USC patients. The preliminary efficacy signal will be confirmed in the dedicated phase 2 study (ZN-c3-004, NCT04814108).

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A method of treating a subject having uterine serous carcinoma (USC), the method comprising administering a therapeutically effective amount of ZN-c3, or a pharmaceutically acceptable salt thereof, to the subject in accordance with a dosing regimen.

2. The method of claim 1, wherein the USC is advanced or recurrent USC.

3. The method of claim 1 or 2, wherein the dosing regimen comprises an oral administration of the ZN-c3.

4. The method of any one of claims 1 to 3, wherein the dosing regimen comprises a periodic dosing phase.

5. The method of claim 4, wherein the periodic dosing phase comprises a daily dose in the range of about 200 mg per day to about 350 mg per day that is fixed for a period of at least three consecutive days.

6. The method of claim 4 or 5, wherein the periodic dosing phase comprises once daily dosing for a period of at least three consecutive days.

7. The method of any one of claims 1 to 6, wherein the dosing regimen comprises an intermittent dosing phase.

8. The method of claim 7, wherein the intermittent dosing phase comprises at least one rest day.

9. The method of claim 7, wherein the intermittent dosing phase comprises at least one change in amount of the ZN-c3 administered to the subject on a daily basis.

10. The method of any one of claims 1 to 9, wherein the dosing regimen comprises five days of dosing of ZN-c3 and two rest days.

11. The method of any one of claims 1 to 10, wherein the therapeutically effective amount of the ZN-c3 administered to the subject is about 200 mg per day.

12. The method of any one of claims 1 to 10, wherein the therapeutically effective amount of the ZN-c3 administered to the subject is ≥300 mg per day.

13. The method of claim 12, wherein the therapeutically effective amount of the ZN-c3 administered to the subject is about 350 mg per day.

14. The method of claim 12, wherein the therapeutically effective amount of the ZN-c3 administered to the subject is about 300 mg per day.

15. The method of any one of claims 1 to 10, wherein the therapeutically effective amount of the ZN-c3 administered to the subject is about 200 mg once daily (QD).

16. The method of any one of claims 1 to 10, wherein the therapeutically effective amount of the ZN-c3 administered to the subject is ≥300 mg once daily (QD).

17. The method of claim 16, wherein the therapeutically effective amount of the ZN-c3 administered to the subject is about 350 mg once daily (QD).

18. The method of claim 16, wherein the therapeutically effective amount of the ZN-c3 administered to the subject is about 300 mg once daily (QD).

19. The method of any one of claims 1 to 10, wherein the therapeutically effective amount of the ZN-c3 administered to the subject is about 100 mg two times a day (BID).

20. The method of any one of claims 1 to 10, wherein the therapeutically effective amount of the ZN-c3 administered to the subject is ≥150 mg two times a day (BID).

21. The method of claim 20, wherein the therapeutically effective amount of the ZN-c3 administered to the subject is about 175 mg two times a day (BID).

22. The method of claim 20, wherein the therapeutically effective amount of the ZN-c3 administered to the subject is about 150 mg two times a day (BID).

23. The method of any one of claims 1 to 22, wherein the subject was previously treated for the USC by a prior therapeutic regimen.

24. The method of claim 23, wherein the prior therapeutic regimen comprises administering a prior USC therapy to the subject.

25. The method of claim 23, wherein the prior USC therapy comprises at least one selected from Carboplatin, Paclitaxel, Bevacizumab, Trastuzumab, Pembrolizumab, Lenvatinib and Doxorubicin.
